(19) United States
(12) Patent Application Publication
(10) Pub. No.: US 2022/0370697 A1
(43) Pub. Date: Nov. 24, 2022

(54) Device and Method to Produce Dialysate

(71) Applicant: THE JOHNS HOPKINS UNIVERSITY, Baltimore, MD (US)

(72) Inventors: Chirag Parikh, Lutherville, MD (US); Derek Michael Fine, Baltimore, MD (US); Mohit Singhala, Baltimore, MD (US); Ryan Bell, Baltimore, MD (US); Brielle Cenci, Pequannock, NJ (US)

(21) Appl. No.: 17/710,522

(22) Filed: Mar. 31, 2022

Related U.S. Application Data
(60) Provisional application No. 63/168,452, filed on Mar. 31, 2021.

(51) Int. Cl. A61M 1/16

(52) U.S. Cl. CPC A61M 1/1656; A61M 2205/3331

(57) ABSTRACT

The present invention provides a novel device and method of generating and bagging dialysate for continuous veno-venous hemodialysis (CVVHD). The procedure involves the use of standard hemodialysis (HD) equipment and the device of the present invention. The device of the present invention is an adapter configured to be used with standard hemodialysis equipment.

### CROSS REFERENCE TO RELATED APPLICATIONS

This application claims the benefit of U.S. Provisional Patent Application No. 63/168,452 filed on Mar. 31, 2021, which is incorporated by reference, herein, in its entirety.

### FIELD OF INVENTION

The present invention relates generally to the field of medical devices. More particularly, the present invention relates to a device and method to produce dialysate.

### BACKGROUND

During the COVID-19 pandemic, it became apparent that major events could result in dialysate shortages. It is therefore desirable to provide a device and method for the production of dialysate.

### SUMMARY

The foregoing needs are met, to a great extent, by the present invention wherein in one aspect, a device for producing dialysate includes an adapter having an outer wall and having a first end and a second end. The outer wall defines an interior lumen that extends between the first and second ends of the adapter. The first end of the adapter is configured to be coupled to dialysis equipment and wherein the second end is configured to be coupled to a dialysate collection bag.

In accordance with an aspect of the present invention, the interior lumen includes couplings for the dialysis equipment and the collection bag. The couplings are threaded couplings. The adapter is formed from a plastic. The adapter is manufactured via three-dimensional printing. The present invention can also include a method of producing dialysate using the adapter of claim 1 to couple the dialysate collection bag to the dialysis equipment.

In accordance with another aspect of the present invention, a method includes generating an adapter having an outer wall and having a first end and a second end, wherein. The outer wall defines an interior lumen that extends between the first and second ends of the adapter. The first end of the adapter is configured to be coupled to dialysis equipment and wherein the second end is configured to be coupled to a dialysate collection bag.

In accordance with yet another aspect of the present invention, the interior lumen includes couplings for the dialysis equipment and the collection bag. The couplings are threaded couplings. The adapter is formed from a plastic. The method includes generating the adapter using three-dimensional printing generating the adapter using a molding process, and/or sculpting the adapter from a block of plastic. The adapter can be formed from one selected from a group of polylactic acid (PLA), nylon, and/or polycarbonate (PC).

### BRIEF DESCRIPTION OF THE DRAWINGS

Further objectives and advantages will become apparent from a consideration of the description, drawings, and examples.

FIGS. 1A-1F illustrate views of an adapter according to an embodiment of the present invention.

FIGS. 2A and 2B illustrate side views of two different embodiments of an adapter according to an embodiment of the present invention.

FIG. 3 illustrates a side view of an adapter, according to the embodiment of the invention illustrated in FIG. 2B.

FIGS. 4A and 4B illustrate image views of the connection between the adapter and the standard dialysis equipment and the collection bag, respectively.

FIG. 5 illustrates an image view of the connection between the adapter and the standard dialysis equipment and the collection bag.

FIG. 6 illustrates a schematic diagram of a complete system for creating and collecting dialysate, according to an embodiment of the present invention.

FIGS. 7A, 7B and 8-16 illustrate image views of a method of producing dialysate according to an embodiment of the present invention.

### DETAILED DESCRIPTION

The presently disclosed subject matter will now be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention provides a novel device and method of generating and bagging dialysate for continuous veno-venous hemodialysis (CVVHD). The procedure involves the use of standard hemodialysis (HD) equipment and the device of the present invention. The device of the present invention is an adapter configured to be used with standard hemodialysis equipment.

A device according to the present invention allows for CVVHD dialysate to be produced by generating it from conventional hemodialysis (HD) machines. Testing of this dialysate demonstrates that its composition is comparable to the CVVHD bags that are available commercially. When the dialysate flow rate is set to 800 mL/min on the HD machine, approximately 400 mL/minute of dialysate can be produced, since the flow is pulsatile.

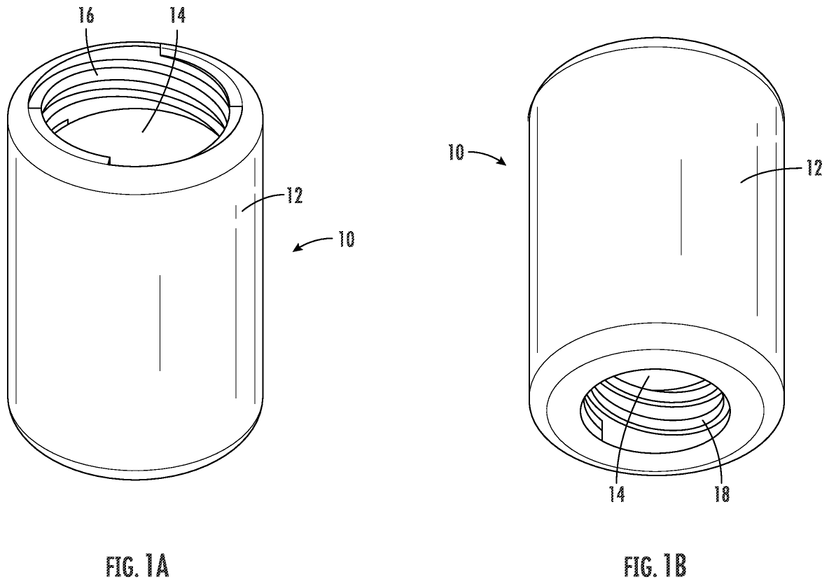

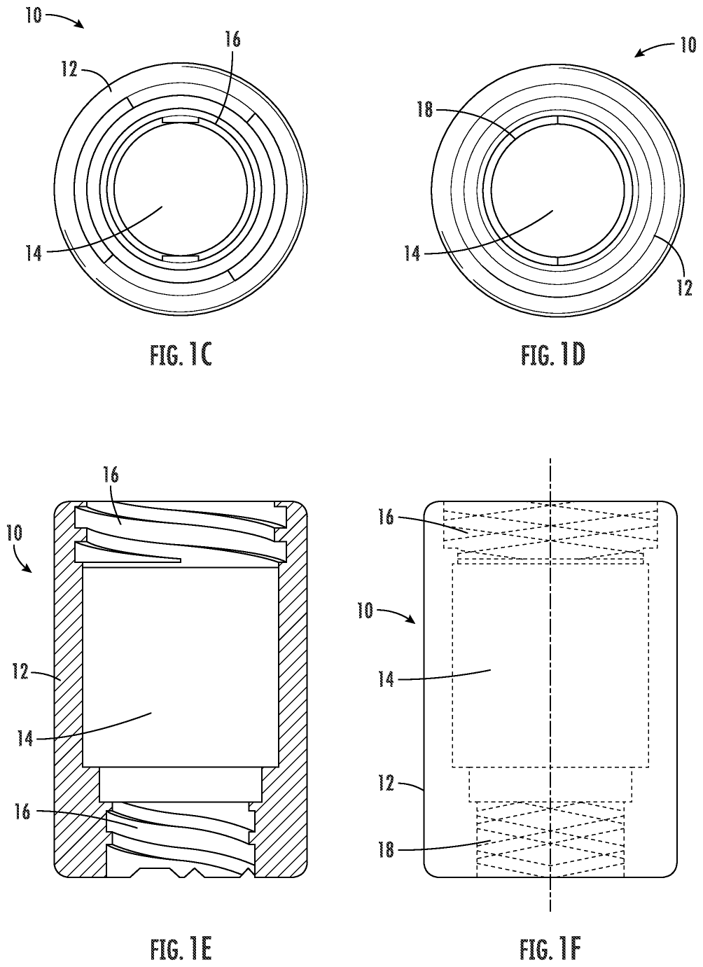

FIGS. 1A-1F illustrate views of an adapter according to an embodiment of the present invention. FIGS. 1A and 1B are perspective views, FIG. 1C is a top-down view, FIG. 1D is a bottom-up view, and FIGS. 1E and 1F are sectional views of the device. The device takes the form of an adapter 10 that couples with standard hemodialysis equipment. The adapter 10 includes an outer wall 12. The outer wall 12 defines an inner lumen 14. The inner lumen 14 includes couplings 16, 18 for coupling the adapter 12 to the standard hemodialysis equipment and to a collection bag. The couplings 16, 18, as illustrated are shown as threaded couplings, but any coupling known to or conceivable to one of skill in the art could be used. The couplings can also be chosen based on compatibility with the corresponding standard hemodialysis equipment and the collection bag. As illustrated in FIGS. 1A-1F, the coupling 16 has a larger diameter that coupling 18 to couple with the standard hemodialysis equipment. The coupling 18 has a smaller diameter that coupling 16 to couple with the collection bag. It should be noted that these configurations are exemplary and not meant to be considered limiting. The adapter and couplings can be configured in any way known to or conceivable to one of skill in the art to be compatible with the standard hemodialysis equipment and the collection bag. The adapter 10 is further configured to maintain a sterile passage for the dialysate to pass through from the standard dialysis equipment into the collection bag.

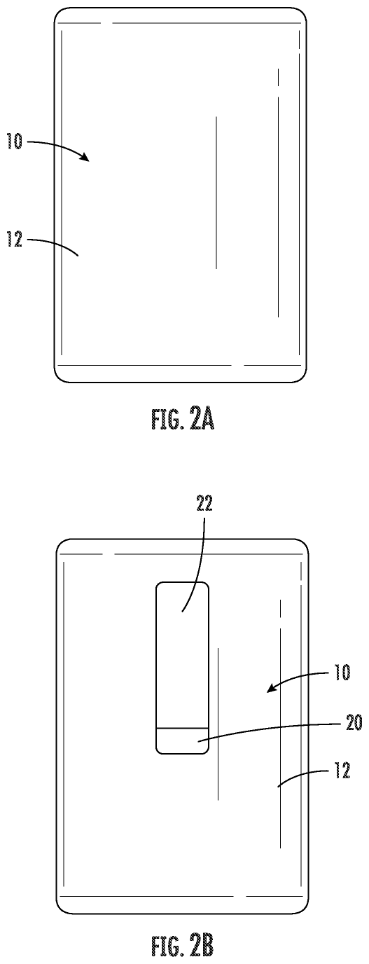

FIGS. 2A and 2B illustrate side views of two different embodiments of an adapter according to an embodiment of the present invention. FIG. 2A illustrates a side view of an adapter according to the device illustrated in FIGS. 1A-1F. FIG. 2A illustrates a side view of an adapter with additional windows for visual inspection of the connection between the standard dialysis equipment and the collection bag. In both embodiments, the adapter 10 includes a wall 12. In FIG. 2B, the wall 12 defines windows 20 and 22, for visualization of the connection between the standard dialysis equipment and the collection bag.

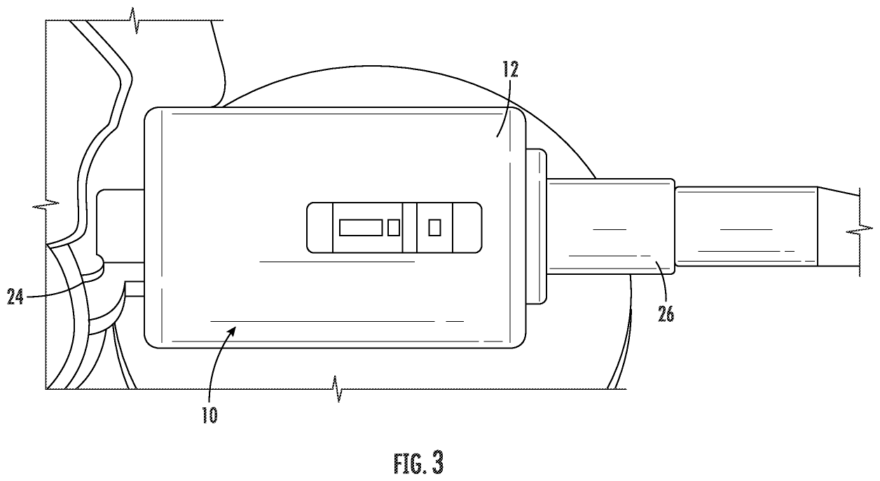

FIG. 3 illustrates a side view of an adapter, according to the embodiment of the invention illustrated in FIG. 2B. FIG. 3 shows the interface between the standard dialysis equipment 24 and the adapter 12 on the left side of the adapter 12. FIG. 3 also shows the interface between the collection bag 26 and the adapter 12 on the right hand side of the adapter 12.

The adapter described above can be three-dimensionally printed, manufactured via molding, sculpting. or any other suitable method for manufacturing the adapter known to or conceivable to one of skill in the art. The adapter can be formed from polylactic acid (PLA), nylon, polycarbonate (PC), or any other suitable material known to or conceivable to one of skill in the art. The material should be sterilizable, and should exhibit low deformation.

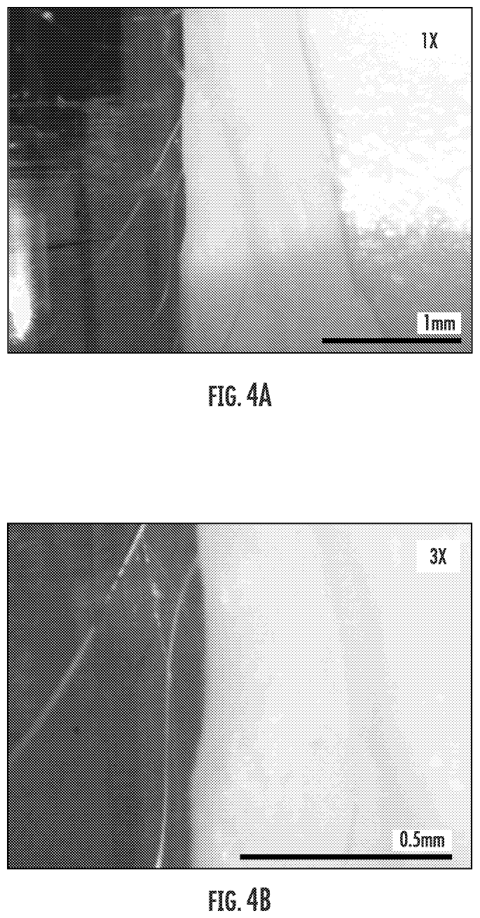

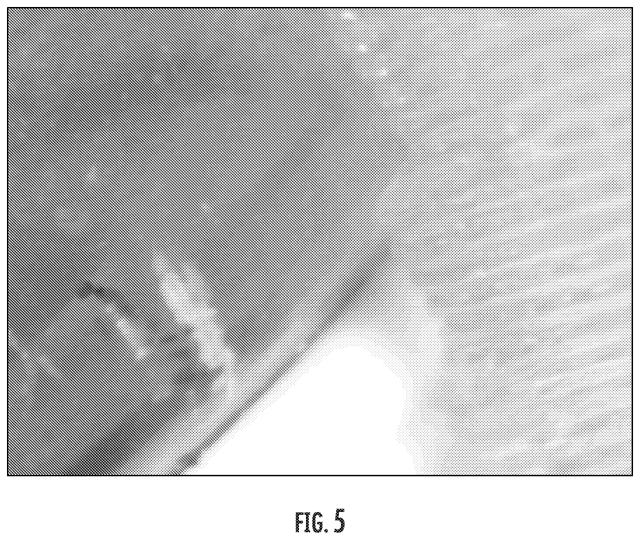

FIGS. 4A and 4B illustrate image views of the connection between the adapter and the standard dialysis equipment and the collection bag, respectively. FIG. 4A illustrates a 1× magnification and FIG. 4B illustrates a 3× magnification. In both FIGS. 4A and 4B, the connection for the standard dialysis equipment is on the left and the connection for the connector of the collection bag is on the right.

FIG. 5 illustrates an image view of the connection between the adapter and the standard dialysis equipment and the collection bag. The image of FIG. 5 is taken through the window defined by the adapter, as illustrated in FIGS. 2B and 3. The top left shows the connection between the standard dialysis equipment and the adapter. The bottom left shows the connector of the collection bag. The right shows the edge of the adapter. As shown in FIG. 5, the adapter has been three-dimensionally printed and the layers of plastic are visible. Based on the images of FIGS. 4A, 4B, and 5, there are no air gaps between the standard dialysis equipment and the connector of the collection bag. Therefore, the dialysate being produced makes no contact with the adapter.

Prototyping and testing was done for three materials: Polylactic Acid (PLA), Nylon, and Polycarbonate (PC). Ultimaker brand filament spools were used for all prints, to ensure compatibility with the Ultimaker 3 Extended printer that was used to manufacture all parts. These spools are widely available and perform consistently well under normal use.

### Materials:

PC: PC has superior mechanical and thermal properties than most common 3D printing materials. The adapter had no visible defects from the use of chlorhexidine swabs or EtO sterilization. But, they were significantly deformed after being autoclaved with a steam cycle. While Polycarbonate is a common material used for medical devices of similar nature, the Ultimaker PC is NOT medical grade and therefore, the adapter should not be assumed to be either biocompatible or to have adequate leaching properties. However, the non-contacting nature of the adapter design should help bypass any biocompatibility concerns, especially if EtO sterilization is performed and chlorhexidine swabs are used. A summary of the PC sterilization tests can be seen below. Tests are currently being performed to determine how many cycles an adapter can go through before it should be changed. Note: All diameters were measured between the threads on both ends. The steam cycle PC samples did not retain their shape and became elliptical on both ends. Failure mode for PC: Formal tests have not been performed on the effects of repeated use of chlorhexidine swabs on a PC adapter. If any visual anomalies are observed, switching out the adapter with a new one is recommended. The adapters of the present invention are also being tested for any visual anomalies.

TABLE 1

| PC | | | Before Sterilization | | | | After Sterilization | | | |
|---|---|---|---|---|---|---|---|---|---|---|
| # | | # of | ID | ID | | | ID | ID | | |
| Sample | Type | Cycles | TPN | Dialyzer | Height | Fit | TPN | Dialyzer | Height | Fit |
| 1 | EtO | 1 | 10.57 | 15.01 | 31.54 | Yes | 10.56 | 15.03 | 31.53 | Yes |
| 2 | EtO | 1 | 10.58 | 15 | 31.53 | Yes | 10.55 | 15.01 | 31.55 | Yes |
| 3 | EtO | 3 | 10.59 | 15.03 | 31.55 | Yes | 10.56 | 15.04 | 31.53 | Yes |
| 4 | EtO | 3 | 10.6 | 14.91 | 31.52 | Yes | 10.6 | 14.94 | 31.55 | Yes |
| 5 | EtO | 7 | 10.61 | 14.97 | 31.53 | Yes | 10.54 | 15.01 | 31.52 | Yes |
| 6 | EtO | 7 | 10.59 | 14.94 | 31.51 | Yes | 10.59 | 15.02 | 31.53 | Yes |
| 7 | Steam | 1 | 10.59 | 14.99 | 31.56 | Yes | NA | NA | 32.31 | No |
| 8 | Steam | 1 | 10.62 | 14.93 | 31.54 | Yes | NA | NA | 32.33 | No |

### Other Materials:

PLA: PLA was only used for prototyping and it is NOT recommended to use PLA for functional parts. PLA is not compatible with most sterilization methods and does not have the adequate mechanical properties to be used for this purpose. However, PLA is a highly popular filament, and is recommended for the testing.

Nylon: Nylon has excellent mechanical properties. It showed significant changes on its surface when wiped with chlorhexidine swabs, but deformed under a single steam cycle in the autoclave. EtO sterilization tests were not performed for Nylon.

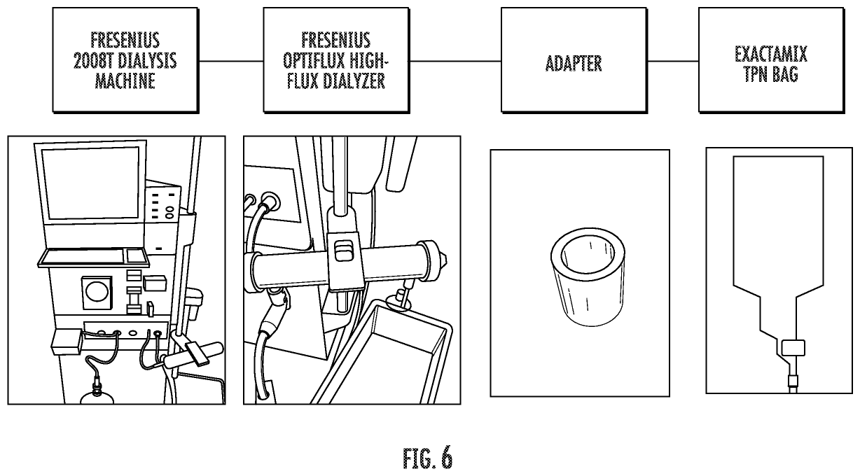

FIG. 6 illustrates a schematic diagram of a complete system for creating and collecting dialysate, according to an embodiment of the present invention. The system includes a standard dialysis machine, a dialyzer, the adapter of the present invention, and a collection bag. The adapter of the present invention is configured or configurable to be used with any dialysis equipment or collection bag known to or conceivable by one of skill in the art. For example, the adapter can be modified to work with any collection bag or container.

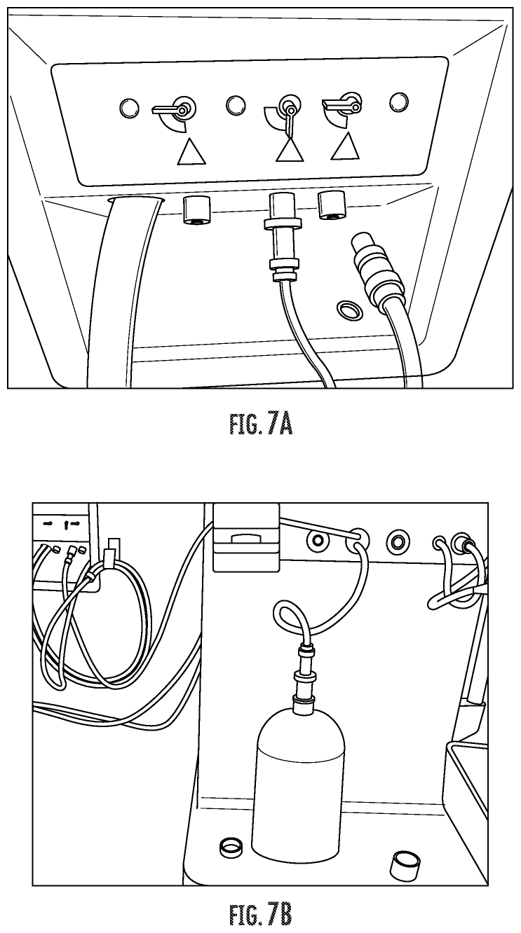

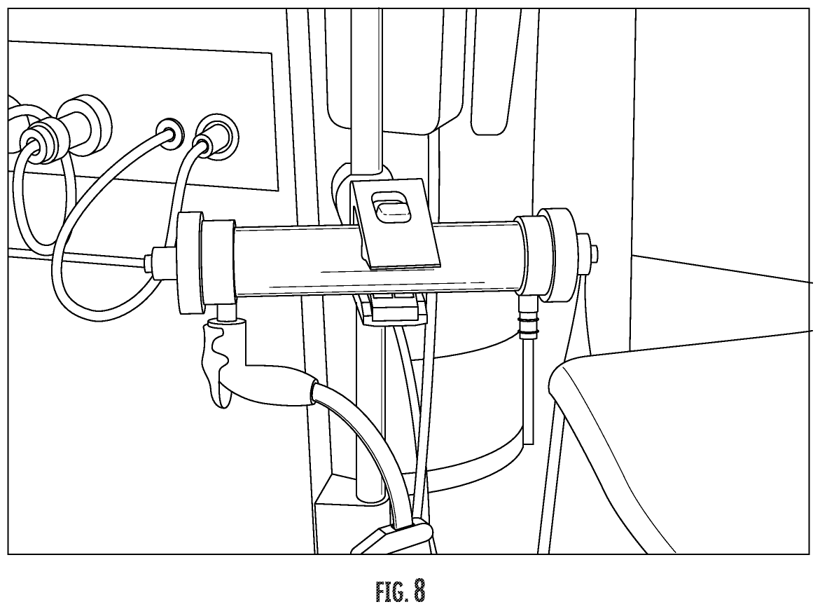

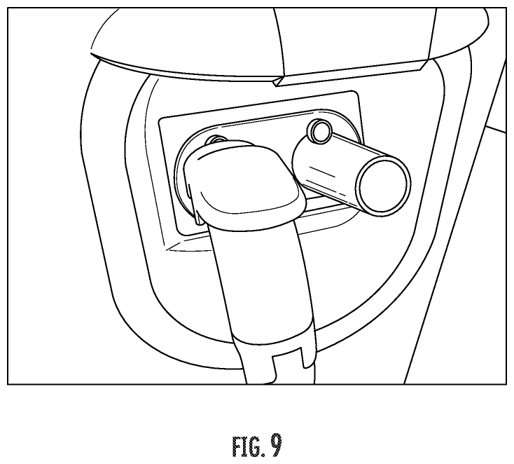

The method of using the adapter with standard dialysis equipment to produce dialysate in collection bags is described further herein with respect to FIGS. 7A, 7B and 8-16. The method described herein is included by way of example and is not meant to be considered limiting. Any method known to or conceivable by one of skill in the art can be used. The following procedure uses a Fresenius 2008T hemodialysis machine to generate and bag dialysate for use in CVVHD treatments. This device is included by way of example and it is noted that any dialysis machine can be used. While the fluid created is safe to use as dialysate, it is not sterile and should under no circumstance be used as infusate.

### Process:

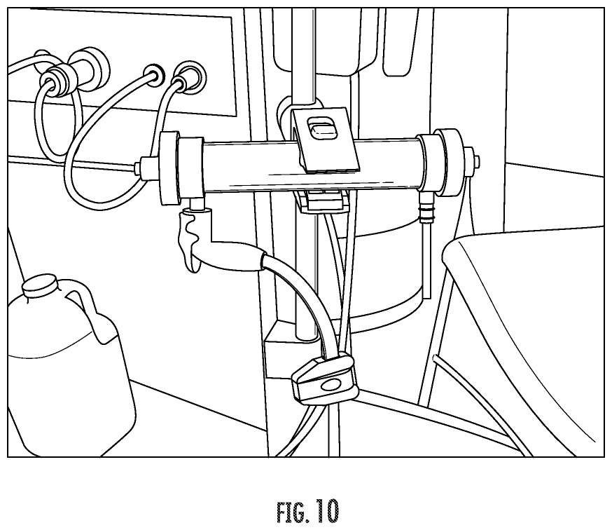

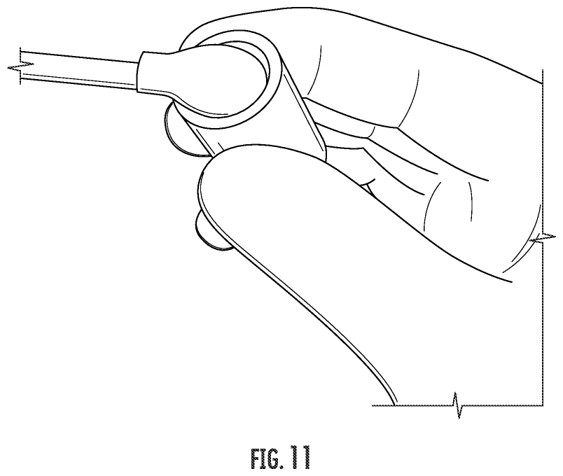

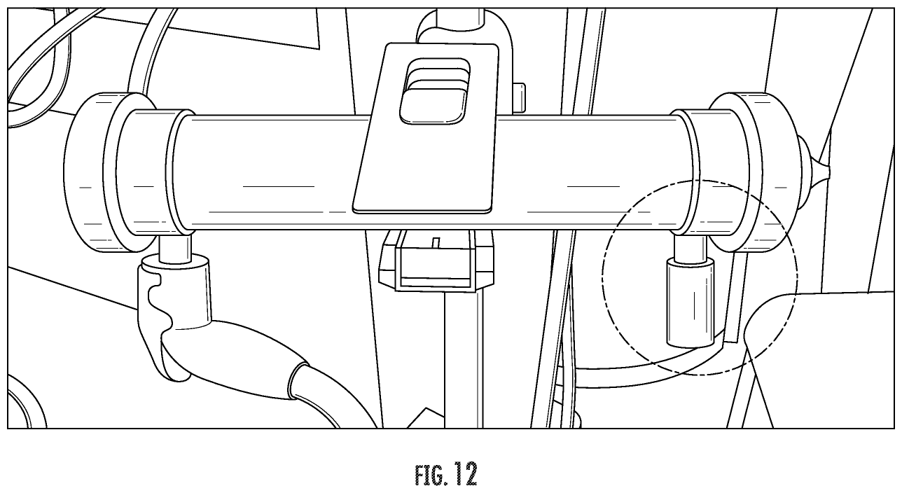

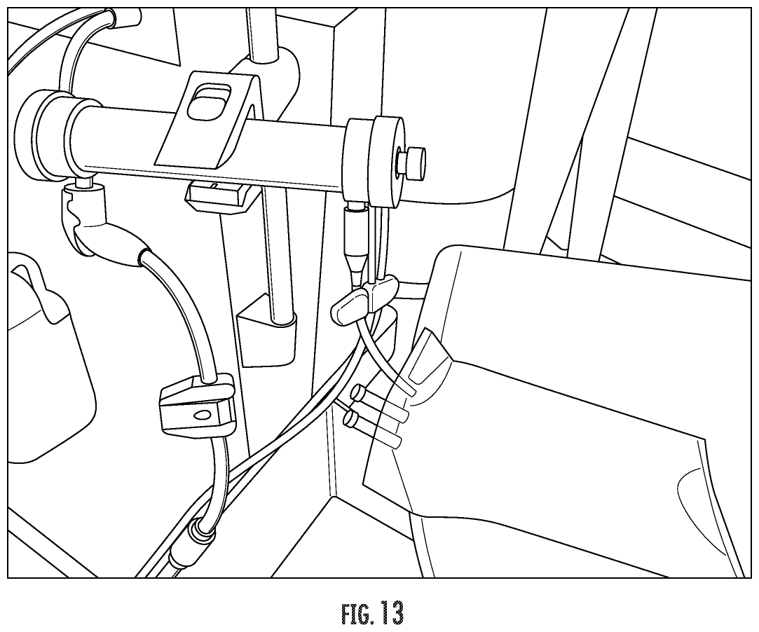

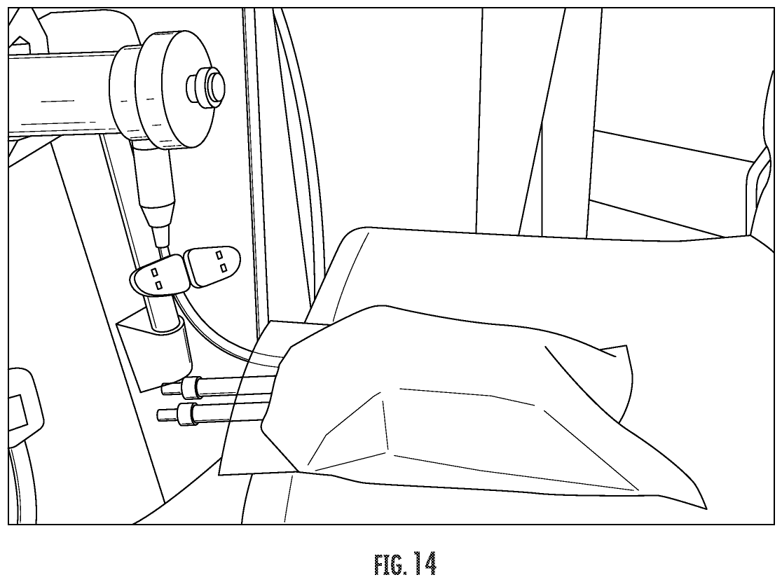

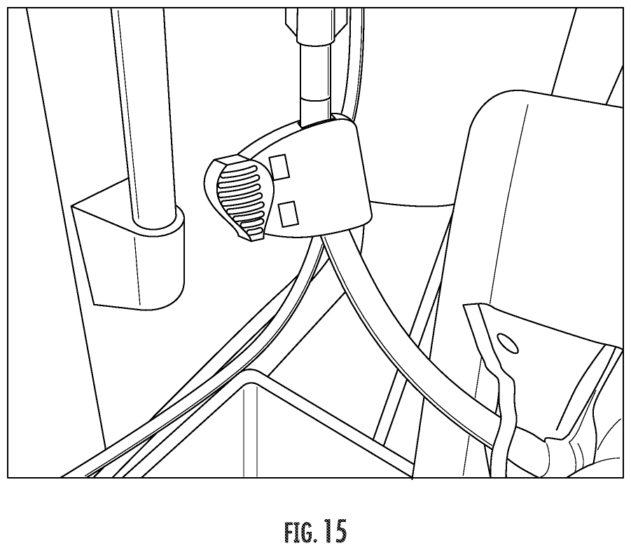

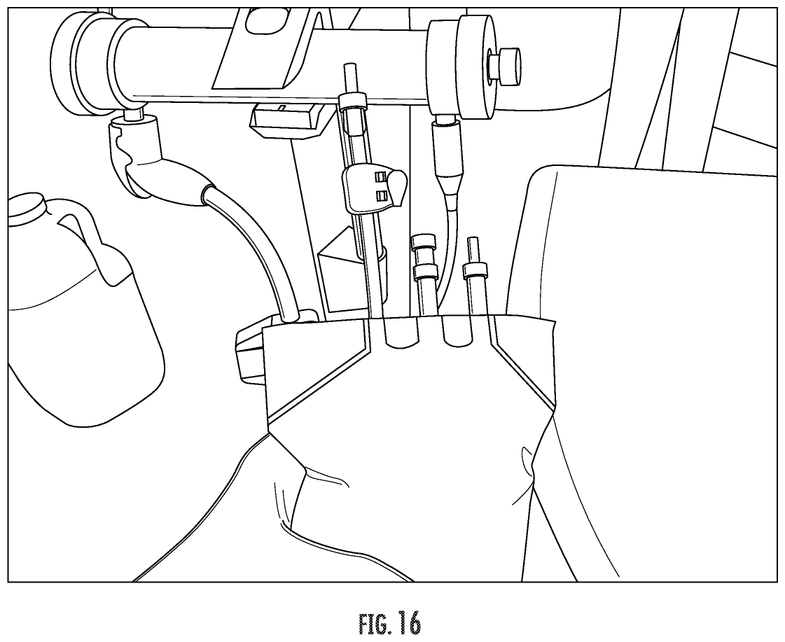

1) Power the machine ON, as illustrated in FIG. 7A
2) Hook up acid connectors and the bicarbonate solution, as illustrated in FIG. 7B
3) Put the machine on dialysis mode (choose the concentrate being used). No bloodlines need to be hooked up.
4) Set the bicarbonate level
5) Run 800 mL/min flow and set preferred temperature. Lower temperature settings allowed us to generate dialysate faster.
6) Wait for machine to reach appropriate stable conductivity (around 13.7-13.9)
7) Once machine has stable conductivity:
   a) Take out blue hansen (shunt interlock), sterilize it with chlorhexidine swabs, and hook it up to the dialyzer inflow port. Remove the cap from the effluent port, as illustrated in FIG. 8.
      i) Avoid moving the dialyzer or the hansen hose (shunt interlock) to prevent the machine from giving TMP error.
      ii) If a TMP error comes on, just clear it, and the machine should continue making dialysate.
   b) Use something that will keep the button pressed inside where the head of the hanson usually hits so the machine doesn't go on bypass mode. A piece tubing that is cut to the right fit is used, as illustrated in FIG. 9.
   c) Close the shunt interlock cover. The machine should make dialysate nonstop but will stop from time to time to cycle, as illustrated in FIG. 10.
8) Once the dialysis machine has been prepped, open the shunt interlock door to stop the flow.
9) Take out an adapter and scrub it with a chlorhexidine swab, as illustrated in FIG. 11.
10) Aseptically attach the larger opening of the adapter to the effluent port of the dialyzer by screwing it on, as illustrated in FIG. 12.
   a) Avoid directly touching the effluent port and the inside of the adapter in order to maintain sterility.
   b) Avoid moving the dialyzer to prevent a TMP error.
11) Aseptically attach the collection bag to the smaller end of the adapter by screwing it on, as illustrated in FIG. 12.
12) The ExactaMix 5000 mL TPN bag is used to collect the dialysate. It is possible to use other bags as may be known to or conceivable to one of skill in the art.
   a) Avoid touching the threads on the bag's fill port and the inside of the adapter.
   b) Make sure that the connection between the TPN bag screw and the adapter is secure, the flange of the TPN bag screw should sit flush with the adapter's flat surface with no room for further travel.
   c) Avoid moving the dialyzer to prevent a TMP error.
13) Close the shunt interlock door to resume flowing dialysate, thus filling the bag. It is advised to place the bag on a table or chair to support the bag's weight as it fills.
   a) Confirm that the bag is filling and monitor for leaks around the adapter, as illustrated in FIG. 13.
14) Once the bag is full, stop the dialysate flow by opening the shunt interlock door, then clamp the bag's tubing. This clamp will help maintain the sterility of the dialysate as it is being removed from the adapter, as illustrated in FIG. 14.
15) Unscrew the full dialysate bag from the adapter to remove it and then put the original cap back on the dialysate bag's screw, as illustrated in FIG. 15.
16) While the adapter is still attached to the dialyzer, use a chlorhexidine swab to scrub both the inside and outside of the adapter.
17) To fill more bags, repeat steps 11-15 with a new bag.

a) Repeated use of the same adapter may result in a loss of sterility. Remember to scrub the adapter with chlorhexidine prior to connecting a new bag.

The adapter of the present invention is configured to connect the dialyzer to a fluid storage bag. In particular, the examples used herein are the Fresenius Optiflux high-flux dialyzer and the Baxter Exactamix TPN bags. Both these devices have male threads on their ends that do not mate with each other. The generally cylindrical adapter of the present invention includes complementary female threads on each end to enable this connection in a way that allows the dialysate to pass from the dialyzer's effluent port directly to the TPN bag without touching the adapter.

In some embodiments the adapter of the present invention is three-dimensionally printed. A preferred process for printing follows. It should be noted that this printing process is included by way of example and is not meant to be considered limiting. Any printing or other manufacturing process known to or conceivable to one of skill in the art can also be used.

### Process:

Print the adapter with the smaller diameter side of the adapter facing the build plate. All the testing and analysis has been performed using the “Ultimaker 3 (Extended)” printer, which uses the Fused Deposition Modeling (FDM) method to create parts. The adapter of the present invention is designed to work with single-extrusion printers and DO NOT need any support material. The Ultimaker family of printers is one option for equipment or any other printers with similar capabilities. The adapter of the present invention are PC sterilizable and PC has superior mechanical properties than most commonly available 3D printing materials. EtO sterilization is recommended for the adapters of the present invention and disinfection with chlorhexidine swabs between uses. Results from the sterilization tests can be found in Table 1. Use the standard latest version of Cura to set up the prints, if using an Ultimaker. The default Ultimaker PC profile on Cura as the baseline. Make the following changes and confirm that the temperatures in the default profile match.
1) Layer Height: 0.15 mm. Print only at a layer height of 0.15 mm or lower to ensure that adapter connects properly. No significant advantage was found in terms of fit or function when printing at 0.1 mm.
2) Printing Temperature: 280 degree C. [Should be default for PC when using Cura]
3) Build Plate Temperature: 107 degree C. [Same as default for PC when using Cura]
4) Infill Percentage: 50%. The quality of the parts at 50% infill is great and has performed well under EtO sterilization with no observable mechanical deformation. Higher infill percentages might increase part strength but may not be necessary under the current use case.
5) Infill Type: Cubic only is used to print the adapters. However, any infill type suited for higher strength should yield similar results.
6) Support Properties [DO NOT use any support or adhesion unless absolutely necessary]
   a) Please print the adapter with the smaller diameter side of the adapter facing the build plate. In this orientation, no additional supports will be needed.
   b) Please uncheck any dual-extrusion options while setting up the print. Most standard support materials are incompatible with Ultimaker PC. No support is needed to print these adapters as such.
   c) Prime blob: If using prime blobs, please make sure that no extra material is carried over from the blob to the print.
   d) Print Adhesion: The adapters have been printing well in the tests with adhesion set to “None”. If the parts are not sticking to the surface, “Rafts” is recommended.

Please DO NOT use glue or any other material to improve print adhesion on these parts to avoid affecting their potential sterility later on.

In order to determine the stability of the dialysate created, chemical analyses were performed to monitor key analyte concentrations over time. At the beginning of the experiment the procedure outlined in section 1 was used to collect two full bags of dialysate in the ExactaMix TPN bags. One was stored in a 4° C. refrigerator and the other was stored at room temperature. Samples were taken at hour 0, 6, 9, 12, and 23 to monitor the stability of the dialysate overtime. Specifically, the concentrations of 8 analytes were taken: calcium, chloride, bicarbonate, glucose, potassium, lactate, magnesium, and sodium. Both bags of dialysate remained reasonably stable for at least 23 hours. In the first 12 hours, no analyte concentration varied from the baseline by more than 5%. At the 23 hour mark, this was true for all analytes except for the refrigerated bicarbonate measurement which varied by about 10%. The full results can be found in Tables 2 and 3, below. The FDA guidelines suggest that generated dialysate should be used within 4 hours, so the generated dialysate will remain stable well past this time point.

TABLE 2
Analyte testing of the dialysate at room temperature (RT). All units in mEg/L.

| Apr. 16, 2020: | | | | | | | | | |
|---|---|---|---|---|---|---|---|---|---|
| Dialysate RT | | | | | | | | | |
| Stability | | | | | | | | | |
| Time | Run | Analyte | | | | | | | |
| Point | Time | Calcium | Chloride | Bicarbonate | Glucose | Potassium | Lactate | Magnesium | Sodium |
| Baseline | 10:21 | 5.2 | 104 | 29.9 | 108 | 2.1 | 0 | 1.29 | 140 |
| (BL) | | | | | | | | | |
| 6 hr | 16:10 | 5.3 | 103 | 31.9 | 106 | 2.2 | 0 | 1.28 | 140 |
| 9 hr | 19:15 | 5.2 | 104 | 31.6 | 109 | 2.1 | 0 | 1.28 | 140 |
| 12 hr | 22:10 | 5.2 | 104 | 32.4 | 104 | 2.2 | 0 | 1.25 | 141 |
| 23 hr | ~9:10 | 5 | 104 | 31.3 | 106 | 2.1 | 0 | 1.32 | 139 |
| Percent | | −3.8 | 0 | 4.7 | −1.9 | 0.0 | N/A | 2.3 | −0.7 |
| Difference | | | | | | | | | |
| (BL-23 | | | | | | | | | |
| hr %) | | | | | | | | | |

TABLE 3
Analyte testing of the dialysate at 4° C. All units in mEg/L.

| Apr. 16, 2020: | | | | | | | | | |
|---|---|---|---|---|---|---|---|---|---|
| Dialysate | | | | | | | | | |
| Refrigerated | | | | | | | | | |
| Stability | | | | | | | | | |
| Time | Run | Analyte | | | | | | | |
| Point | Time | Calcium | Chloride | Bicarbonate | Glucose | Potassium | Lactate | Magnesium | Sodium |
| Baseline | 10:23 | 5.1 | 104 | 29.8 | 107 | 2.2 | 0 | 1.25 | 140 |
| (BL) | | | | | | | | | |
| 6 hr | 16:05 | 5.2 | 103 | 32.1 | 107 | 2.2 | 0 | 1.3 | 140 |
| 9 hr | 19:10 | 5.1 | 104 | 32.7 | 109 | 2.1 | 0 | 1.23 | 140 |
| 12 hr | 22:10 | 5.1 | 104 | 31.3 | 107 | 2.1 | 0 | 1.26 | 140 |
| 23 hr | ~9:10 | 5 | 103 | 32.9 | 106 | 2.1 | 0 | 1.33 | 140 |
| Percent | | −2.0 | −1 | 10.4 | −0.9 | −4.5 | N/A | 6.4 | 0.0 |
| Difference | | | | | | | | | |
| (BL-23 | | | | | | | | | |
| hr %) | | | | | | | | | |

At the beginning of the experiment the above procedure was used to collect two full bags of dialysate in the ExactaMix TPN bags. One was stored in a 4° C. refrigerator and the other was stored at room temperature. The following is the testing procedure used.
1) Two samples were received (room temperature and 4° C.) at two hour intervals over 6 intervals.
2) Total of six room temperature and six 4° C. samples were received.
3) All fluids were centrifuged for 5 minutes at 3,000 RPM before plating.
4) The supernatant was removed leaving approximately 0.25 ml to re-suspend the sediment/pellet prior to inoculation.

5) Using a sterile, individually wrapped pipette, one drop of the fluid was plated to blood agar and chocolate agar plates for the aerobic culture and one drop to a brucella blood agar plate for the anaerobic culture. Then, plates were streaked for isolation.
6) Aerobic plates were incubated in a CO2 incubator at 37° C.±2° C. and anaerobic plates were incubated in anaerobic condition at 37° C.±2° C.
7) Aerobic plates were checked at 24 and 48 hrs.
8) Anaerobic plates were checked at 48 and 96 hrs.

The results of the culture tests showed that there was no growth on any of the plates throughout the experiment, suggesting that the bagged dialysate fluid was safe for use. While the results are promising, the test does not prove that the fluid is sterile. Therefore, it is only suggested to use this method if non-sterile dialysate is appropriate. This method is not recommended for creating infusate, as sterility cannot be guaranteed. It is encouraged that microbiology tests are performed intermittently to instill confidence that this method continues to produce dialysate that is safe to use.

Visual inspection of the adapter was performed under normal operation and also the Dialyzer-TPNbag interface was analyzed under a microscope. These tests were performed on a special test version of the adapter of the present invention. The test adapter is dimensionally equivalent but has two diametrically opposite slots that enable visual inspection of the dialyzer-collection bag interface through the naked eye and a microscope.

To further check for leaks, the collection bag was filled with water and blue watercolor was added to enhance visibility under the microscope. Then the water was allowed to flow under gravity by raising the bag above the connection to check for leaks. This was repeated twice. One slot was used to obtain the images and the other was used to check for any leaks during the tests. A chromatography strip was placed under the connection, facing the diametrically opposite slot to collect any fluid, if it leaked. After the test, another chromatography strip was pressed against the outside of the dialyzer-collection bag screw's interface, through the slot, to check for leaks as well. Both chromatography papers were found to be dry,

Visual inspections of the slotted test adapters were also performed when connected to the dialysis machine and were monitored for leaks. No leaks were observed under normal operation. The non-contacting nature has only been verified in a limited setting, under normal operation. At high pressure, or in cases where the procedure is not followed, extra care should be taken to monitor for leaks. The adapter of the present invention is not expected to fail under normal operation. The adapter is designed to be intuitive to use, however, the user must ensure that both, the effluent port of the dialyzer and the male screw of the TPN bag are completely screwed in. If not done correctly, the fluid may either leak over the threads of either the connections and seep out or, if there is a large enough gap, the fluid might make contact with the inner wall of the adapter before entering the bag. To ensure a proper fit, it is recommended to screw the adapter over the dialyzer effluent port first. When it can no longer move, screw in the collection bag until the flange of the TPN bag's male screw is flush with the flat surface of the adapter. It is recommend that users to support their bags on a flat surface (like a chair or a table) as they are filled. While the adapters of the present invention can support the weight of a fully filled 1L collection bag, this is not recommended. The weight of the filled bags can cause tensile stress in the adapter and stretch it over time, jeopardizing its non-contacting nature.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A device for producing dialysate comprising:
an adapter having an outer wall and having a first end and a second end, wherein the outer wall defines an interior lumen that extends between the first and second ends of the adapter;
wherein the first end of the adapter is configured to be coupled to dialysis equipment and wherein the second end is configured to be coupled to a dialysate collection bag.

2. The device of claim 1 wherein the interior lumen comprises couplings for the dialysis equipment and the collection bag.

3. The device of claim 2 wherein the couplings are threaded couplings.

4. The device of claim 1 wherein the adapter is formed from a plastic.

5. The device of claim 1 wherein the adapter is manufactured via three-dimensional printing.

6. A method of producing dialysate using the adapter of claim 1 to couple the dialysate collection bag to the dialysis equipment.

7. A method comprising:
generating an adapter having an outer wall and having a first end and a second end, wherein the outer wall defines an interior lumen that extends between the first and second ends of the adapter;
wherein the first end of the adapter is configured to be coupled to dialysis equipment and wherein the second end is configured to be coupled to a dialysate collection bag.

8. The method of claim 7 wherein the interior lumen comprises couplings for the dialysis equipment and the collection bag.

9. The method of claim 8 wherein the couplings are threaded couplings.

10. The method of claim 7 wherein the adapter is formed from a plastic.

11. The method of claim 7 further comprising generating the adapter using three-dimensional printing.

12. The method of claim 7 further comprising generating the adapter using a molding process.

13. The method of claim 7 further comprising sculpting the adapter from a block of plastic.

14. The method of claim 7 wherein the adapter is formed from one selected from a group consisting of polylactic acid (PLA), nylon, and polycarbonate (PC).
